(19) United States
(12) Patent Application Publication
(10) Pub. No.: US 2008/0033527 A1
(43) Pub. Date: Feb. 7, 2008

(54) METHODS AND SYSTEMS FOR MONITORING AN ENDOPROSTHETIC IMPLANT

(76) Inventors: Anthony Nunez, Beachwood, OH (US); Harry Rowland, Peoria, IL (US)

Correspondence Address: Patrick W. Rasche;Armstrong Teasdale LLP, Suite 2600, One Metropolitan Square, St. Louis, MO, 63102, US

(21) Appl. No.: 11/773,295

(22) Filed: Jul. 3, 2007

Related U.S. Application Data
(60) Provisional application No. 60/819,534, filed on Jul. 7, 2006.

(51) Int. Cl. A61F 2/06

(52) U.S. Cl. 623/1.13; 623/1.15

(57) ABSTRACT

A prosthetic implant includes a graft having a wall defining a passage. A plurality of sensors are integrated with the graft. The sensors are configured to detect at least one structural characteristic of the graft. A power source is operatively coupled to the sensors and configured to provide power to the sensors.

### CROSS REFERENCE TO RELATED APPLICATIONS

This application claims the benefit of U.S. Provisional Application No. 60/819,534, filed Jul. 07, 2006.

### BACKGROUND OF THE INVENTION

This invention relates generally to implantable medical devices or prosthetic implants, and, more particularly, to an endoprosthesis and a method of monitoring an endoprosthetic implant in a body lumen.

Aortic aneurysms are a common cause of death. Specifically, an aortic aneurysm involves an outpouching or dilation in an arterial wall due to a weakening, loss of elasticity, and overall degeneration in the arterial wall caused by plaque build up in the artery. If left untreated, an aortic aneurysm may expand to a point of rupture potentially causing death. Generally, aortic aneurysms are treated with an open surgery; however, not every patient is a candidate for such a surgery. Moreover, an open surgery has a greater chance for complications, involves at least one substantial incision, and/or requires an extended hospital stay for the patient.

An alternative to open surgery involves endoluminally by-passing the aneurysm using an endoprosthetic graft or stent. Specifically, the endoprosthesis is inserted into the artery and positioned to block or exclude the aneurysmal sac. Resultantly, blood is allowed to flow through the artery without entering and expanding the aneurysmal sac. The insertion of an endoprosthesis is minimally invasive, requires shorter hospital stays, and has a lower probability of complication.

As such, an endoprosthesis provides a desirable alternative to open surgery; however, at least some known endoprosthetics may fail after being inserted in the body lumen. Specifically, a leak or “endoleak” may occur at any time after the insertion of the endoprosthesis. Four types of endoleaks are commonly known to occur. A first type of endoleak occurs when there is a persistent amount of blood flow around the endoprosthesis because of an inadequate seal between the endoprosthesis and the artery wall. A second type of endoleak occurs when a retroflow of blood enters the aneurysmal sac from lumbar arteries, the inferior mesenteric artery, or collateral vessels. A third type of endoleak may occur when there is a tear in the endoprosthesis allowing blood to flow therethrough. Finally, a fourth type of endoleak may occur due to a permeability or porosity of the endoprosthesis, wherein blood flows through the wall of the endoprosthesis.

To monitor the success of the endoprosthesis, patient follow-ups are commonly scheduled after surgery. During a follow-up, patients are often subjected to arteriography, contrast-enhanced spiral CT, ultrasonography X-ray, and/or intravascular ultrasound. Because such follow-up procedures are costly, invasive, and minimally effective, at least some known endoprosthetics are designed with sensors that allow pressure and blood flow in and around the aneurysmal sac to be monitored. However, at least some known endoprosthetics equipped with sensors do not account for thrombus, a solid or semi-solid cholesterol build-up that may occur within the aneurysmal sac. Specifically, thrombus results in an inaccurate reflection of the forces being transmitted to the aneurysmal sac.

### BRIEF DESCRIPTION OF THE INVENTION

In one aspect, a method of monitoring an endoprosthesis for insertion into a body lumen is provided. The method includes implanting the endoprosthesis into the body lumen to exclude an aneurysmal sac in a vascular region and monitoring characteristics of the endoprosthesis using a plurality of sensors coupled thereto, wherein monitoring the characteristics includes monitoring at least one of an endoprosthesis wall tension, an endoprosthesis circumference, and an endoprosthesis diameter.

In another aspect, a modular endoprosthesis for implantation in a body lumen to exclude an aneurysmal sac in a vascular region is provided. The endoprosthesis includes a plurality of sensors to monitor characteristics of the endoprosthesis, wherein the characteristics include at least one of an endoprosthesis wall tension, an endoprosthesis circumference, and an endoprosthesis diameter.

In a further aspect, a system for monitoring characteristics of an endoprosthesis is provided. The system includes a power source and a modular endoprosthesis for implantation in a body lumen to exclude an aneurysmal sac in a vascular region. The endoprosthesis includes a plurality of sensors to monitor characteristics of the endoprosthesis, wherein the characteristics include at least one of an endoprosthesis wall tension, an endoprosthesis circumference, an endoprosthesis diameter, a pressure on the luminal surface, and a pressure on the exterior surface. The endoprosthesis also includes at least one transmitter to transmit signals indicative of the characteristics. The system also includes a device external to the body lumen to receive the transmitted signals.

In a further aspect, a prosthetic implant is provided. The prosthetic implant includes a graft having a wall defining a passage and a plurality of sensors integrated with the graft. The plurality of sensors are configured to detect at least one structural characteristic of the graft. A power source is operatively coupled to the plurality of sensors and configured to provide power to the plurality of sensors.

In a further aspect, a prosthetic implant is provided. The prosthetic implant includes a plurality of flexible leaflets cooperatively movable between an open position defining a passage and a closed position. At least one sensor is integrated within at least one leaflet of the plurality of leaflets. At least one sensor is configured to detect at least one structural characteristic of the plurality of leaflets. A power source is operatively coupled to at least one sensor and configured to provide power to at least one sensor.

### BRIEF DESCRIPTION OF THE DRAWINGS

FIG. 1 is a schematic view of an endoprosthesis positioned within a body lumen;

FIG. 2 is a schematic cross-sectional view of a capacitive pressure sensor that may be used with the endoprosthesis shown in FIG. 1;

FIGS. 3-8 schematically show a method for manufacturing pressure sensors suitable for use with the endoprosthesis shown in FIG. 1;

FIG. 9 is a schematic view of an exemplary system used to monitor the endoprosthesis shown in FIG. 1;

FIG. 10 is a bottom perspective view of an exemplary implantable medical device including sensors;

FIG. 11 is a top perspective bottom view of the implantable medical device shown in FIG. 10; and

FIG. 12 is a perspective view of an alternative exemplary implantable medical device.

### DETAILED DESCRIPTION OF THE INVENTION

The present invention provides a system and method for monitoring structural characteristic values of a medical device implanted within a patient and/or physiological parameter concentrations, values and/or conditions within the patient. The system includes an implantable prosthetic device that is positioned within the patient's body, such as within a body lumen including, without limitation, a blood vessel, or within a cavity defined by an organ, such as within one or more chambers of the patient's heart. The device includes one or more sensors configured to sense or detect one or more structural characteristic values of the device including, without limitation, stress, strain, tension, compression, extension, elongation, expansion, migration and other displacement values including a change in diameter, circumference, length and/or width of the device. Additionally or alternatively, the sensors are configured to sense or detect one or more physiological parameter concentrations, values or conditions within the device and/or the surrounding environment including, without limitation, pressure, temperature, flow velocity, humidity and/or pH level. Further, the sensors may include at least one position sensor, tactile sensor, accelerometer and/or microphone.

In the exemplary embodiment, the sensors are operatively coupled to an external monitoring system, such as an external computing system, configured to receive representative signals transmitted by the sensors, manipulate the transmitted signals and provide a diagnosis of the patient to facilitate caring for the patient based at least partially on the transmitted signals. The data, as represented by the signals transmitted by the sensors, is provided to the integrated computing system, which then applies system software to confirm, model and/or analyze the structural integrity and position of the device and/or the physiological environment in which the device is implanted. The sensors may be operatively coupled to and/or in signal communication with other components of the system using electrical, electronic or electromagnetic signals including, without limitation, optical, radio frequency, digital, analog or other signaling configurations. By monitoring the structural characteristic values for the implanted device and/or the patient's physiological parameter concentrations, values and/or conditions, the system facilitates effectively treating the patient.

The present invention is described below in reference to its application in connection with and operation of an implantable medical device or prosthetic implant and, more particularly, to an endoprosthesis, such as a stent graft, a heart valve device, and a shunt, such as a cerebral spinal fluid (CSF) shunt. However, it should be apparent to those skilled in the art and guided by the teachings herein provided that the invention is likewise applicable for use with suitable medical applications incorporating implantable medical devices including, without limitation, other grafts, stents, heart valve devices and shunts, filters such as Greenfield filters, coils, orthopedic devices such as hip and knee replacement systems, spinal implants, and other prosthetic implants suitable for insertion within the patient's ear, eye, nose, mouth, larynx, esophagus, blood vessel, vein, artery, lymph node, breast, stomach, pancreas, kidney, colon, rectum, ovary, uterus, gastrointestinal tract, bladder, prostate, lung, brain, heart or other organ of the patient, for treatment of infection, glaucoma, asthma, sleep apnea, gastrointestinal reflux, incontinence, hydrocephalus, heart disease and defects, and other conditions or diseases. Further, the system and/or one or more components of the system are likewise applicable to industrial and military applications including, without limitation, deep sea diving, flying, mining, and other applications wherein the subject is exposed to pressure variations, for example.

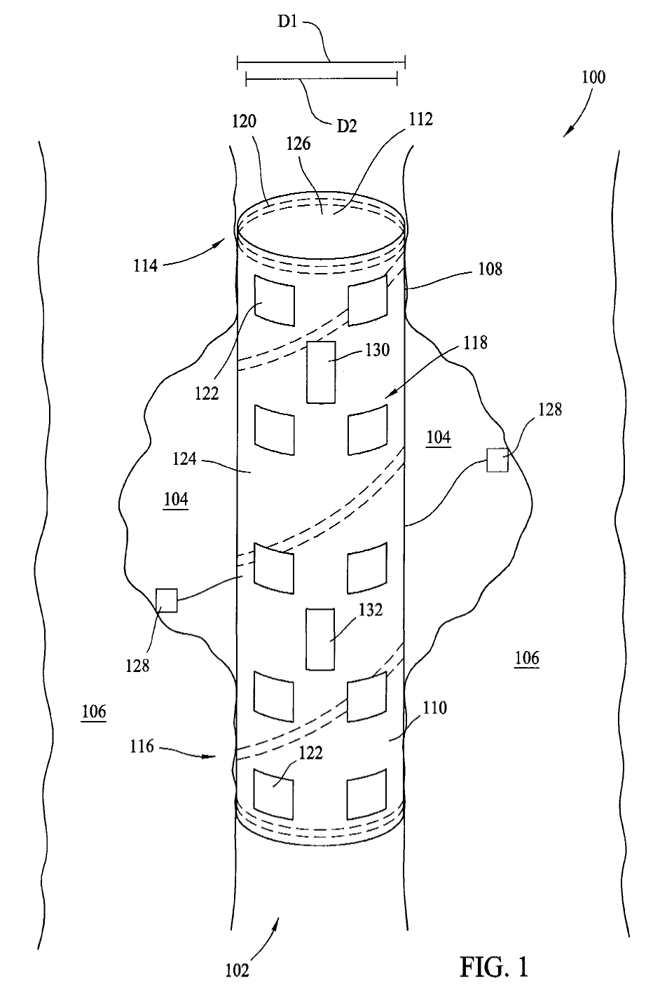

FIG. 1 is a schematic view of a prosthetic implant, namely a stent graft or endoprosthesis 100, inserted into a body lumen 102. More specifically, in one embodiment, endoprosthesis 100 is positioned within body lumen 102 to exclude an aneurysmal sac 104. Aneurysmal sac 104 is formed by an outpouching or dilation in a wall 106 of body lumen 102. Aneurysmal sac 104 may be categorized as an abdominal aortic aneurysm (AAA), a thoracic aortic aneurysm (TAA), or an aneurysm in one of the iliac arteries, for example. Endoprosthesis 100 may be utilized to treat any aneurysmal sac 104 existing in any body lumen.

Referring further to FIG. 1, in one embodiment, endoprosthesis 100 includes a graft 108 having a wall 110 defining a passage 112. In one embodiment, graft 108 is fabricated of a suitable biocompatible material including, without limitation, a polyester, expanded polytetrafluoroethylene (ePTFE) or polyurethane material and combinations thereof. It is apparent to those skilled in the art and guided by the teachings herein provided that graft 108 may include any suitable biocompatible synthetic and/or biological material, which is suitable for implanting within the injured or diseased blood vessel. In this embodiment, graft 108 is substantially tubular having an outer diameter D₁, an inner diameter D₂, an outer circumference and an inner circumference.

Graft 108 at least partially defines a first end 114, an opposing second end 116 and a midportion 118 of endoprosthesis extending between first end 114 and second end 116. Endoprosthesis 100 is positioned within body lumen 102 such that first end 114 and second end 116 form a suitable seal with body lumen wall 106 to prevent or limit blood flow between endoprosthesis 100 and body lumen wall 106 into aneurysmal sac 104. Midportion 118 extends along a length of aneurysmal sac 104 to exclude aneurysmal sac 104 from body lumen 102. Passage 112 extends between first end 114 and second end 116 such that fluid, namely blood, flowing through body lumen 102 is channeled through passage 112 to prevent fluid flow into aneurysmal sac 104. In a particular embodiment, endoprosthesis 100 includes graft 108 having one or more branched portions each having a substantially tubular configuration and defining an outer diameter, an inner diameter, an outer circumference and an inner circumference.

In a particular embodiment, a stent 120 is positioned with respect to graft 108. Referring to FIG. 1, stent 120 is positioned within graft 108. Stent 120 is formed of a suitable biocompatible material including, without limitation, a metal, alloy, composite or polymeric material and combinations thereof. In one embodiment, stent 120 is formed of a shape-memory material, such as a nitinol material. Other suitable materials for forming stent 120 include, without limitation, stainless steel, stainless steel alloy and cobalt alloy. It is apparent to those skilled in the art and guided by the teachings herein provided that stent 120 may include any suitable biocompatible synthetic and/or biological material, which is suitable for implanting within the injured or diseased blood vessel.

As shown in FIG. 1, stent 120 is positioned within graft 108 and is movable between a radially compressed configuration and a radially expanded configuration to support graft 108 within body lumen 102, for example, with respect to aneurysmal sac 104. In a particular embodiment, an induction coil, as described in greater detail below, is coupled to stent 120.

Endoprosthesis 100 is positioned within body lumen 102 using surgical methods and delivery apparatus for accessing the surgical site known to those skilled in the art and guided by the teachings herein provided. Such surgical methods and delivery apparatus may be used to place endoprosthesis 100 within the vasculature and deliver endoprosthesis 100 to a deployment site. The apparatus may include various actuation mechanisms for retracting sheaths and where desired, inflating balloons of balloon catheters. Endoprosthesis 100 may be delivered to the deployment site using any suitable method and/or apparatus. One suitable method includes a surgical cut down made to access the femoral artery. The catheter is inserted into the femoral artery and guided to the deployment site using fluoroscopic or intravascular imaging, where endoprosthesis 100 is then deployed. An alternative method includes percutaneously accessing the blood vessel for catheter delivery, i.e., without a surgical cutdown. An example of such a method is described in U.S. Pat. No. 5,713,917, the disclosure of which is incorporated herein by reference.

In one embodiment, endoprosthesis 100 is delivered in a radially compressed configuration through a surgically accessed vasculature to the desired deployment site. In this embodiment, endoprosthesis 100 is loaded into a catheter (not shown) in a generally linear position and held in a radially compressed configuration by a sheath to retain endoprosthesis 100 in the compressed configuration to prevent or limit undesirable contact between endoprosthesis 100 and wall 106 and, more specifically, between graft wall 110 and wall 106, as endoprosthesis 100 is delivered to the deployment site. With a distal end of the catheter sheath located at the deployment site, the catheter sheath is retracted to deploy endoprosthesis 100. In a particular embodiment, radio-opaque markers (not shown) are coupled to or integrated with endoprosthesis 100, such as coupled to or integrated with an outer surface of graft 108, at selected or desired locations to facilitate orientating endoprosthesis with respect to aneurysmal sac 104 utilizing a suitable imaging device prior to deployment. For example, the radio-opaque markers may be positioned with respect to one or more expandable portions and/or one or more semi-cylindrical portions, particularly in a branched endoprosthesis, to properly position and orient endoprosthesis 100 at the deployment site.

For applications related to the treatment of an AAA, the endoprosthesis is orientated such that the contralateral limb is positioned to face in a general direction to allow cannulation of the open end. The contralateral limb is then deployed and cuffed extensions are then added proximally and distally or at the junctions to create a sealed endoprosthesis. For applications related to treatment of a TAA, the tubular or branched endoprosthesis is oriented such that the semi-cylindrical portion is aligned with the smaller radius curved portion of the vessel. The proximal and distal ends are determined by angiograms or intravascular ultrasound, which delineate the optimal seal zone, while delineating the related major and minor branches, such as the left subclavian artery. The tubular or branched endoprosthesis expands to bias or urge the endoprosthesis toward an interior surface of the body lumen to fixedly engage the endoprosthesis with the interior surface of the body lumen upstream and downstream of the aneurysm site or diseased portion. The expandable sections expand or contract to flexibly conform to the anatomy of the vessel. The expanding and contracting may, for example, be by folding and unfolding a corrugated section, or by stretching or relaxing the endoprosthesis material.

Total coverage of a TAA may require a plurality of endoprosthesis, such as two, three, four or five endoprosthesis. In one embodiment, the endoprosthesis are delivered to fit the aneurysm starting with the smallest graft being placed proximally followed by placement of the larger grafts within the smaller graft so that the radial force exerted by the larger graft creates the necessary resistance to migration.

Similarly, the smaller grafts may be placed distally first and then the larger grafts added proximally such that the coverage is built from the distal end toward the proximal end. In an alternative embodiment, the TAA endoprostheses may be placed proximally and distally with the final interconnecting pieces added to completely exclude the remaining midportion.

Hooke's law describes strain in the following equation:

$$δ = \frac{P ℓ}{AE}$$

Where:
P=force producing extension of bar (lbf)
l=length of bar (in.)
A=cross-sectional area of bar (in.²)
d=total elongation of bar (in.)
E=elastic constant of the material, called the Modulus of Elasticity, or Young's Modulus (lbf/in.²)
The quantity E, the ratio of the unit stress to the unit strain, is the modulus of elasticity of the material in tension or compression and is often called Young's Modulus.

The quantity, E, the ratio of the unit stress to the unit strain, is the modulus of elasticity of the material in tension or compression and is often called Young's Modulus. Thus, for example, with a metal wire of a stent temporarily displaced, a sensor measures the displacement of the metal wire to determine the strain and, thus, the wall tension within the endoprosthesis and/or the stent. The sensor provides real time feedback during implantation to facilitate accurately positioning the endoprosthesis at or within the aneurysm site. The wall tension of the endoprosthesis and/or the stent applied to the aortic wall provides real time feedback indicating a maximum wall tension within the endoprosthesis and/or the stent, while at the same time there is a simultaneous drop in the sac pressure as well as angiographic confirmation.

It has been described that the electrical energy can be derived from the body of a human utilizing either the kinetic motion of the body or the heat lost to the ambient surroundings. In one embodiment, the kinetic energy derived from a motion of the graft as the graft expands into the aneurysm sac, thereby expanding the wire stent components against a magnetically coupled circuit generates the necessary μohms required for powering the device. Alternatively, the piezoelectric change from the incorporation of a piezoelectric film, such as Polyvinylidene Difluoride (PVDF), into the graft design at selected portions of the graft located in the most pulsatile area serves as a potential integral power source for the sensors.

As shown in FIG. 1, one or more sensors 122 are coupled to or integrated with endoprosthesis 100. In one embodiment, a plurality of sensors 122 are positioned on endoprosthesis 100 to provide an integrated network of sensors 122. In the exemplary embodiment, sensors 122 are positioned with respect to an exterior wall surface 124 and/or an interior wall surface 126 of graft 108. In a particular embodiment, sensors 122 are positioned to allow variability in a choice of sensing. Any suitable configuration of the network of sensors 122 may be provided in alternative embodiments. Sensors 122 may include one or more capacitive pressure sensors, piezoresistors, such as a Wheatstone bridge, and/or any suitable sensor for measuring structural characteristic values of endoprosthesis 100, including structural characteristic values of graft 108 and/or stent 120, and/or physiological parameter concentrations, values and/or conditions. In one embodiment, sensors 122 are fabricated using a suitable micro-electromechanical systems (MEMS) technology.

In the exemplary embodiment, one or more sensors 122 are configured to measure a pressure associated with endoprosthesis 100. By measuring pressures within endoprosthesis 100 and manipulating signals generated by sensors 122 corresponding to or representative of the pressure, characteristics of endoprosthesis 100 can be monitored and analyzed. In one embodiment, sensors 122 are positioned with respect to interior wall surface 126 and/or exterior wall surface 124 and configured to measure a wall tension, an inner and/or outer wall diameter, and/or an inner or outer wall circumference. These measured characteristics are used to monitor endoprosthesis 100 and, more particularly, to monitor potential problems or complications with endoprosthesis 100.

To increase operational reliability, in one embodiment sensors 122 are distributed at an aortic proximal seal point and/or a distal seal point and/or at a junction of modular components within endoprosthesis 100. Additionally or alternatively, sensors 122 are distributed substantially along a length of endoprosthesis 100 to increase a probability of detecting an endoleak. In this embodiment, endoprosthesis 100 includes several rows of sensors 122 positioned proximally, at a midpoint, and distally along endoprosthesis 100. Within the sensor rows, a number of sensors 122 positioned circumferentially about endoprosthesis 100 are activated at a time of interrogation. If one sensor 122 fails, a replacement or redundant sensor 122 adjacent to or near the failed sensor 122 is activated at a different frequency. In an alternative embodiment, failure of one sensor 122 automatically activates the adjacent sensor 122 such that only a limited number of frequencies are utilized.

In one embodiment, endoprosthetic wall tension is measured and utilized to determine and monitor a change in a relationship between endoprosthesis 100 and body lumen wall 106 that may be indicative of an endoleak and/or another potential condition, problem or complication with endoprosthesis 100. In a particular embodiment, tension in endoprosthesis 100 is determined by a fit of endoprosthesis 100 against body lumen wall 106. With endoprosthesis 100 positioned within body lumen 102, midportion 118 experiences a greater tension than first end 114 and/or second end 116 due to a difference in blood pressure between aneurysmal sac 104 and body lumen 102. If an endoleak or other condition or complication occurs, tension within endoprosthesis 100 increases causing a decrease in a ratio of tension between midportion 118 and first end 114 and/or second end 116. By detecting the ratio change, potential problems or complications with endoprosthesis 100 may be avoided or minimized.

Further, a change in the relationship between endoprosthesis 100 and body lumen wall 106 may be determined by a change in outer diameter D₁, inner diameter D₂ and/or the endoprosthesis circumference. In one embodiment, an increase in a size of aneurysmal sac 104 results in displacement or expansion, such as radially outward, of the endoprosthetic wall and, thus, an increase in outer diameter D₁, inner diameter D₂ and/or the endoprosthesis circumference. By measuring outer diameter D₁, inner diameter D₂ and/or the endoprosthesis circumference, structural changes in body lumen wall 106 may be detected such that any potential problems or complications with endoprosthesis 100 are identified.

In alternative embodiments, at least one sensor 122 is configured to measure various other attributes of endoprosthesis 100 including, without limitation, a temperature of endoprosthesis 100, motion such as migration and/or displacement of endoprosthesis 100, a position of endoprosthesis 100 within body lumen 102, a radial force associated with endoprosthetic implantation and an accuracy of endoprosthetic implantation. More specifically, a temperature measurement may be indicative of an infection at the implantation site, motion and position of endoprosthesis 100 may be indicative of a faulty seal, and radial force and accuracy measurements are utilized to ensure a proper seal during implantation. In a further embodiment, sensors 122 are configured to measure attributes, such as physiological parameter values, of aneurysmal sac 104 in conjunction with measurements related to endoprosthesis 100. One or more sensors 122 may be coupled to or integrated with exterior wall surface 124 or may be operatively coupled to endoprosthesis to extend into aneurysmal sac 104 to facilitate measuring the physiological parameter values.

In one embodiment, sensors 122 are configured to measure a force, such as a radial force, that endoprosthesis 100 applies to body lumen wall 106. Additional sensors 122 are configured to measure a position of endoprosthesis 100, a sac pressure and/or a blood pressure. The relationship of these measured attributes and ratios thereof are monitored and/or analyzed to predict a potential failure of endoprosthesis 100 that may result in a Type I endoleak. In an alternative embodiment, one or more sensors 122 are configured to measure endoprosthesis position, wall tension and/or sac pressure within branched endoprostheses to monitor a potential of Type II and/or Type III endoleaks.

In a further embodiment, the endoprosthesis position and sac pressure measurements are used in conjunction with a CAT scan, CT, MRI or Ultrasound based technology to obtain anatomic data that can be integrated with real time physiological data obtained from endoprosthesis 100. For example, an anatomical scan provides information related to the aneurysmal sac size that, when compared to the measured attributes of endoprosthesis 100, is useful in detecting and predicting future endoleaks. Additionally, the information is useful in predicting a potential success of endoprosthesis 100. Moreover, in one embodiment, medical imaging technology provides structural information related to kinking or infolding of endoprosthesis 100. Such information, used with endoprosthetic and sac pressure measurements, allows a pressure reading at or near an endoleak. Further, the ability to integrate graft position, graft wall tension, and sac pressure with medical imaging facilitates providing more reliable, less expensive and/or simplified patient follow-ups.

Sac pressure and graft wall tension may be used in conjunction with fluoroscopic equipment to obtain real time measurements during implantation of endoprosthesis 100 to facilitate accurate placement of endoprosthesis 100 within body lumen 102.

In a further embodiment, one or more sensors 122 are utilized to measure at least one constituent within a fluid flowing through endoprosthesis 100, namely blood. The constituents measured may include, without limitation, oxygen, enzymes, proteins and nutrients. In an alternative embodiment, one or more sensors 122 are configured to detect a kinking, folding and/or enfolding of endoprosthesis 100, which may lead to a structural failure of endoprosthesis 100. Additionally or alternatively, one or more sensors 116 measure an electrical potential of endoprosthesis 100.

In one embodiment, one or more sensors 122 are integrally coupled to or integrated within graft 108. In a particular embodiment, sensors 122 are covered by a thin layer of graft material. Sensors 122 are configured to detect or sense at least one structural characteristic of graft 108, such as a graft implant position, a wall stress, a wall strain, a wall tension, an outer wall circumference, an inner wall circumference, an outer wall diameter, an inner wall diameter and/or a graft temperature. At least one sensor 122 is positioned within exterior wall surface 124 and/or at least one sensor 122 is positioned within interior wall surface 126. Further, sensors 122 may be configured to detect an intraluminal blood pressure, an intravascular blood pressure, a sac pressure and/or an aortic blood pressure. Sensors 122 are integrated within wall 110 and configured to facilitate laminar flow at corresponding exterior wall surface 124 or interior wall surface 126. In one embodiment, sensors 122 are integrally configured about graft 108 in a helical pattern, a linear pattern, a star pattern, or a circumferential pattern to facilitate monitoring an environment within which endoprosthesis 100 is positioned, such as within aneurysmal sac 104.

In a further embodiment, at least one independent sensor 128, i.e., a sensor that is not integrally coupled to graft 108, is operatively coupled to a power source, as described in greater detail below, and configured to detect or sense a portion of aneurysmal sac 104, such as an aneurysm sac wall. Independent sensors 128 may be positioned at the time of deployment of endoprosthesis 100 or may positioned after endoprosthesis deployment utilizing a translumbar approach. The translumbar approach requires a small French catheter that allows the passage of a small pressure sensor that is monitored in GPS manner. This technique is referred to as a Graft Position Sensor System. Sensors 122 and/or sensors 128 may include at least one piezoresistive sensor and/or at least one capacitive sensor. Further, sensors 122 and/or sensors 128 may be energized electromagnetically.

In one embodiment, a power source 130 and a transmitter 132 are operatively coupled, such as in electrical communication with, endoprosthesis 100. Transmitter 132 is configured to transmit signals to a receiving device representative of the measured structural values and characteristics of endoprosthesis 100 and/or the physiological parameter values for the environment within which endoprosthesis is implanted. In the exemplary embodiment, the receiving device is located externally with respect to the patient's body. The external receiving device includes a receiver, a display such as an LCD display, a CPU and/or any other device suitable for receiving, measuring, analyzing and/or displaying signals representative of measurements detected by sensors 122 and/or sensors 128 and/or generated data corresponding to the measurements. Power source 130 is configured to provide an electrical current through sensors 122, 128 and transmitter 132. In the exemplary embodiment, power source 130 creates a piezoelectrical current from a movement of fluid through endoprosthesis 100, a pulsatile movement of endoprosthesis 100, and/or an application of any suitable material to create a piezoelectrical current. In an alternative embodiment, described in further detail below, power source 130 is located externally with respect to the patient's body. In this embodiment, sensors 122, 128 are in signal communication with an external transmitter and receiver. Sensors 122, 128 transmit signals representative of a structural characteristic of endoprosthesis. Data corresponding to the transmitted signals is gathered and complied to monitor graft wall tension, graft position, graft diameter, sac pressure and aortic blood pressure, for example.

In a particular embodiment, power source 130 includes a radio frequency induction coil operatively coupled to sensors 122, 128. The induction coil includes a planar coil, a spiral coil, a spiral coil having a ‘z’ configuration, or a vertical coil configuration. In this embodiment, the induction coil is coupled to stent 120, such as wrapped about at least a portion of stent 120.

In one embodiment, sensors 122 are deployed as a separate system. In this embodiment, separate sensors 122 occupy a unique space. Methods or techniques for deploy sensors 122 include deployment utilizing a small French catheter left behind after the modular graft pieces are properly positioned within the body lumen. The catheters may be positioned through a separate stick site adjacent an endograft introducer. In a particular embodiment, sensors 122 may be pushed out in a coil configuration. For example, a coil system includes sensors 122, which are introduced with a coil to promote thrombosis of the aneurysmal sac if there is an apparent endoleak.

Alternatively, sensors 122 are deployed as a sheet of sensors in a linear configuration or in a spiral configuration. The sheet of sensors may be deployed along with the endograft body and limb as a separate system. During deployment of the sheet, sensors 122 are rolled or, if sensors 122 have suitably small dimensions, in a “string of beads” configuration. In this embodiment, the sheet of sensors is unsheathed with a snap mechanism at a base to facilitate controlling the string.

In a further alternative embodiment, sensors 122 are joined by a nitinol wire and pushed out by a pusher from a back end. The wire including sensors 122 is held along a length of the wire with a mechanism that is configured to break with torsional stress. Alternatively, a cutting mechanism is used to break the connection between the string and the delivery system. The cutting mechanism may include an “over the wire” system or a monorail system.

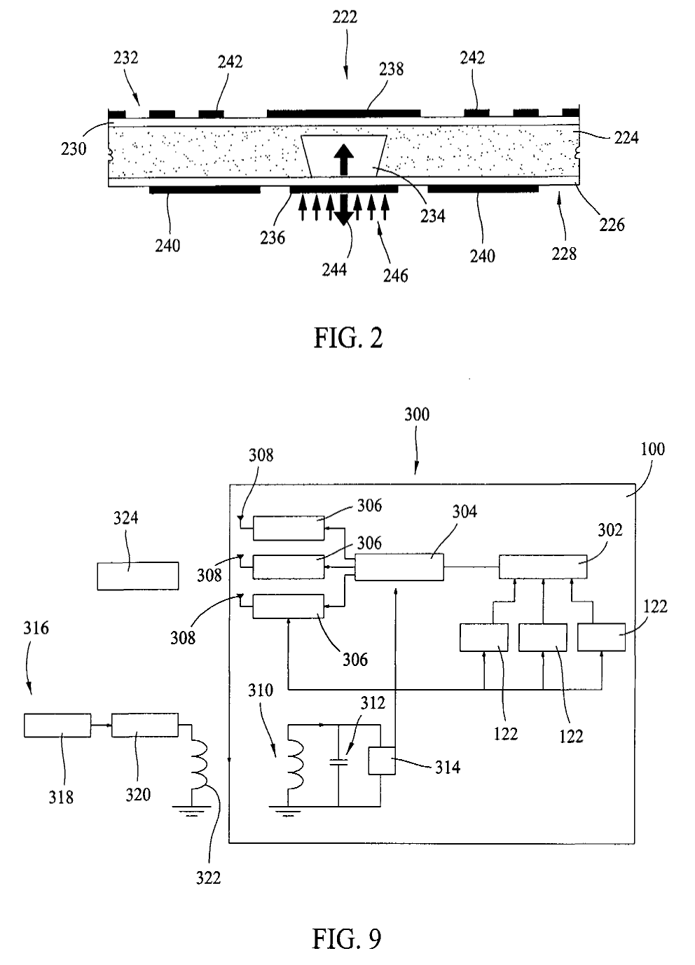

In the exemplary embodiment, the network of sensors 122 includes one or more capacitive pressure sensors. FIG. 2 is a schematic cross-sectional view of a capacitive pressure sensor 222 suitable for use with the network of sensors 122 coupled to or integrated with endoprosthesis 100. In an alternative embodiment, any suitable piezoelectric or piezoresistive pressure sensor may be utilized in cooperation with endoprosthesis 100. Pressure sensor 22 includes a core 224 having a dielectric substrate, such as silicone. A flexible dielectric membrane 226 is coupled to a first or lower surface 228 of pressure sensor 222 and an insulating film 230 is coupled to an opposing second or upper surface 232 of pressure sensor 222. In the exemplary embodiment, dielectric membrane 226 includes silicone oxide and silicone nitride. Pressure sensor 222 defines a cavity 234 formed within core 224. A first or lower capacitor plate 236 is positioned on lower surface 228 and a second or upper capacitor plate 238 is positioned on upper surface 232. Lower capacitor plate 236 and upper capacitor plate 238 are aligned with cavity 234. At least one ground plane 240 is also positioned on lower surface 228 and at least one inductor 242 is positioned on upper surface 232.

Pressure sensor 222 is positioned on endoprosthesis 100 such that changes in luminal or exterior pressure will cause a deformation of pressure sensor 222, as indicated by arrows 244 in FIG. 2. More specifically, forces indicated by arrows 244 acting on pressure sensor 222 bend or deflect pressure sensor 222 about or with respect to cavity 234. The deformation of pressure sensor 222 causes a change in the distance separating capacitor plates 236 and 238. The change in distance separating capacitor plates 236 and 238 changes the capacitance of pressure sensor 222. The resonant frequency (f) of the pressure sensor 222, the inductance (L) of the pressure sensor 222, and the capacitance (C) of the of the pressure sensor 222 can be input into the equation:

$$f = \frac{1}{2 π \sqrt{LC ( p )}}$$

to determine a pressure (p) within the endoprosthetic wall. As described above, by knowing at least one pressure on the endoprosthetic wall, various properties or characteristics of endoprosthesis 100 can be determined. As such, endoprosthesis 100 is monitored to detect a potential problem or complication with endoprosthesis 100 and prevent or minimize any undesirable or harmful effects on the patient associated with the detected problem or complication.

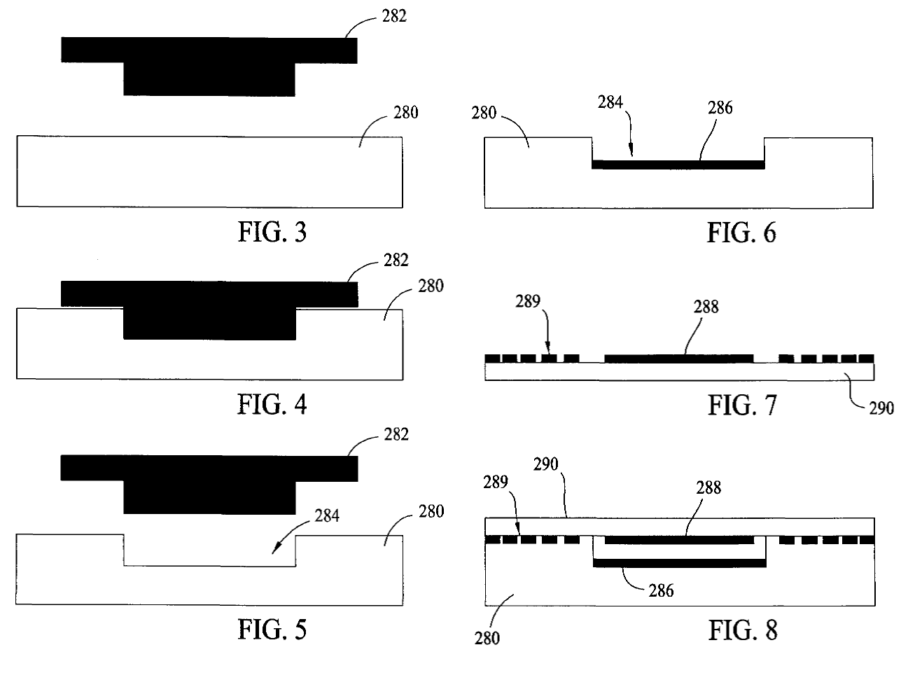

FIGS. 3-8 schematically show a method for manufacturing a pressure sensor 222. A polymer substrate 280 is provided. Polymer substrate 280 may include a non-porous or low porosity polymer, such as polytetrafluorethylene, expanded polytetrafluoroethylene, other fluoropolymers, or any suitable polymer known to those skilled in the art and guided by the teachings herein provided. A master mold 282 is positioned with respect to polymer substrate 280 and pressed into polymer substrate 280, as shown in FIG. 4, to mold or define a cavity 284 within polymer substrate 280, as shown in FIG. 5. Alternatively, cavity 284 may be formed by a suitable process including, without limitation, lithography and chemical etching, ink jet printing, and laser writing.

A pattern of electrically conducting material including a first capacitor plate 286 is layered or deposited on a surface of polymer substrate 280 within cavity 284, as shown in FIG. 6. As shown in FIG. 7, a pattern of electrically conducting material including an inductor 289 electrically connected to capacitor plate 288 is layered onto a second polymer substrate 290. Polymer substrate 290, including patterned capacitor 288 and inductor 289, is then attached to polymer substrate 280 to seal cavity 284, wherein polymer substrate 280 and polymer 290 are axially aligned, as shown in FIG. 8, to form a wireless pressure sensor in polymer with polymer substrate 290 directly above cavity 284 including a membrane that is movable with respect to or toward polymer substrate 280 in response to a change in an external condition.

In one embodiment, polymer substrate 280 and polymer substrate 290 are coated with an additional layer of non-porous or low-porosity material on one or more surfaces such that when attached, polymer substrate 280 and polymer substrate 290 form a hermetically sealed cavity 284. Polymer substrate 290 may be attached to polymer substrate 280 through a variety of processes including, without limitation, adhesive bonding, laminating, and laser welding. In one embodiment, inductor 289 on polymer substrate 290 is electrically connected to capacitor plate 286 on polymer substrate 280 during the attachment process.

In further embodiments, the external surface of pressure sensor 222 may be textured with a controlled topography consisting of features of size ranging from 10 nm-100 μm such that the properties of blood flow near the sensor surface are modified. Patterning the surface of the sensor can modify the coagulation properties to reduce endothelialization and reduce the risk of thrombosis or embolism. Patterning the surface of the sensor can also modify the flow properties of blood near the surface, promoting or reducing slip near the surface to alter the laminar or turbulent characteristics of the flow. The controlled topography may also form small wells that may be filled with a slow release polymer that has been impregnated with an anitmetabolite substance that inhibits cell division, such as Tacrolimus or Sirolimus. The filled wells may then be covered with a porous polymer layer to allow the time-controlled release of drugs. In further embodiments, an external surface of pressure sensor 222 may be coated with a deactivated heparin bonded material for anti-coagulation or antimetabolite coatings.

In an alternative embodiment, pressure sensor 222, as described in FIGS. 3-8, is fabricated in rigid substrates including fused silica, glass, or high resistivity silicon. The cavities in the rigid substrates are formed via wet or dry chemical etch processes. The surfaces are patterned with electrically conducting material in a similar manner to the patterning on polymer substrates. The rigid substrates may be attached by a variety of processes including, without limitation, fusion bonding, anodic bonding, laser welding, and adhesive sealing.

FIG. 9 is a schematic view of an exemplary system 300 used to monitor endoprosthesis 100. System 300 includes a plurality of devices coupled to or integrated with endoprosthesis 100 and a plurality of devices located externally with respect body lumen 102. System 300 includes a plurality of sensors 122 electronically coupled to and in signal communication with an analog to digital converter 302. Although three sensors 122 are shown in FIG. 9, it should be apparent to those skilled in the art and guided by the teachings herein provided that system 300 may include any suitable number of sensors 122 coupled to or integrated with endoprosthesis 100. Sensors 122 may include one or more capacitive pressure sensors 222, as described above, and/or any suitable piezoelectric or piezeoresistive sensor. Referring further to FIG. 9, system 300 also includes a microcontroller 304 electronically coupled to and in signal communication with analog to digital converter 302 and also coupled to one or more radiofrequency identification tags 306, each having an antenna 308. System 300 may include any suitable number of radiofrequency identification tags 306. In a particular embodiment, system 300 includes a radiofrequency identification tag 306 for each sensor 122. An inductor 310 is electronically coupled to a capacitor 312 and a ground plane 314. Ground plane 314 is electronically coupled to each sensor 122, each radiofrequency identification tag 306 and microprocessor 304.

A power source 316 is provided outside body lumen 102. Power source 316 includes an oscillator 318 electronically coupled to an amplifier 320 and an inductor 322. Further, a radiofrequency identification reader 324 is also provided outside body lumen 102.

During operation, a magnetic coupling between inductor 310 and inductor 322 generates an alternating current that is channeled to and powers sensors 122, microcontroller 304 and radiofrequency identification tags 306. Sensors 122 detect and measure pressure within endoprosthetic 100, as described above, and transmit alternating current signals to analog to digital converter 302, wherein the alternating current signals are converted to corresponding digital signals. The digital signals are transmitted to microcontroller 304 and radiofrequency identification tags 306, wherein each digital signal is provided a unique code. The codes are transmitted through antennas 308 to radiofrequency identification reader 324 and the codes are decoded such that the signals can be read by and/or viewed on an integrated monitoring device (not shown), such as an integrated external computing system including a display screen. The signals are processed by the integrated external computing system to monitor and/or analyze properties or characteristics of endoprosthesis 100, as well as physiological parameters within endoprosthesis and/or within the surrounding environment, such that endoprosthesis 100 is monitored externally to detect a real or potential problem or complication with endoprosthesis 100.

In an alternative embodiment, one or more implanted microprocessors are configured to monitor structural properties or characteristics of endoprosthesis 100 including, without limitation, an endoprosthesis wall tension, a position of the endoprosthesis within a body lumen, and/or physiological parameter values of an aneurysmal sac. The implanted microprocessor is operatively coupled to endoprosthesis 100 and in signal communication with sensors 122 to facilitate monitoring the structural characteristics and/or physiological parameter values. Alternatively, the structural characteristics of endoprosthesis 100 and/or the physiological parameter values of the aneurysmal sac may be measured and/or monitored externally using an office based unit or by an ultrasound, CAT scan or MRI based unit fixed, mobile, or otherwise. In yet another embodiment, a handheld device, such as, but not limited to, a cell phone, PDA or a combination thereof, may be utilized by a patient to gather the internal data, which is then downloaded telephonically, over the internet or transmitted wirelessly to a monitoring datapoint.

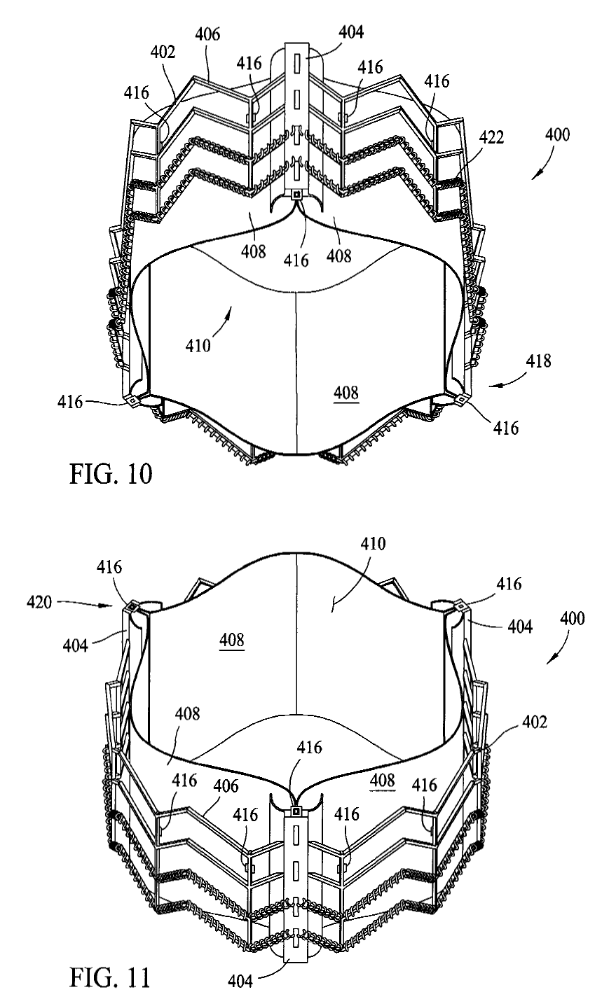

FIGS. 10-12 are perspective views of an implantable medical device or prosthetic implant, namely a heart valve device 400, for treating a defective or damaged heart valve. Heart valve device 400 may be suitable for replacing a mitral valve, an aortic valve, a tricuspid valve or a pulmonary valve. Heart valve device 400 is positionable within the respective valve annulus and coupled to the valve rim. More specifically, heart valve device 400 includes a frame 402 that is positioned within the valve annulus and coupled to the valve rim using a suitable coupling mechanism, such as a suture. Additionally or alternatively, frame 402 includes a plurality of anchoring members (not shown), such as hooks, barbs, screws, corkscrews, helixes, coils and/or flanges, to properly anchor heart valve device 400 within the annulus.

Frame 402 is formed of a suitable biocompatible material including, without limitation, a metal, alloy, composite or polymeric material and combinations thereof. In one embodiment, frame 402 is formed of a shape-memory material, such as a nitinol material. Other suitable materials for forming frame 402 include, without limitation, stainless steel, stainless steel alloy and cobalt alloy. It is apparent to those skilled in the art and guided by the teachings herein provided that frame 402 may include any suitable biocompatible synthetic and/or biological material, which is suitable for implanting within the injured or diseased blood vessel. Frame 402 includes a plurality of generally parallel support members 404 and a plurality of cross-members 406 coupled between adjacent support members 404 to collectively define an outer periphery of heart valve device 400. Heart valve device 400 further includes a plurality of flexible leaflets 408 coupled between adjacent support members 404, as shown in FIGS. 10-12. Although heart valve device 400 shown in FIGS. 10-12 includes three leaflets 408, in alternative embodiments, heart valve device 400 may include any suitable number of leaflets 408. Leaflets 408 are configured to move cooperatively to open and close the respective valve opening 410 to facilitate controlling blood flow through the valve opening.

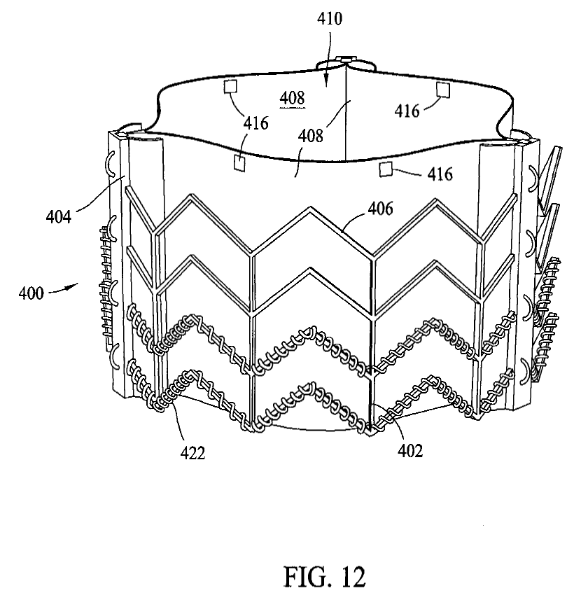

As shown in FIGS. 10 and 11, one or more sensors 416 are coupled to frame 402 at selected locations on heart valve device 400 to facilitate monitoring the structural properties or characteristics of heart valve device 400 and/or the physiological parameter values within the surrounding environment of the patient's heart. In one embodiment, sensors 416 are evenly spaced about a periphery of heart valve device 400. Additionally or alternatively, one or more sensors 416 are integrated with at least one leaflet 408 at selected locations to facilitate monitoring the structural properties or characteristics of heart valve device 400 and/or the physiological parameter values within the surrounding environment of the patient's heart, as shown in FIG. 12. In the exemplary embodiment, sensors 416 are substantially identical to or similar to sensors 122 and may include one or more pressure sensors 222, as described above. In a particular embodiment, a sensor 416 is coupled to a first end 418, as shown in FIG. 10, and/or an opposing second end 420, as shown in FIG. 11, of one or more support members 404. Additionally or alternatively, at least one sensor 416 is coupled to one or more cross-members 406, as shown in FIG. 10. In one embodiment, sensors 416 are fabricated using a suitable micro-electromechanical systems technology, such as described above in reference to sensors 122.

In one embodiment, heart valve device 400 includes flexible leaflets 408 cooperatively movable between an open position defining a passage and a closed position. In one embodiment, each leaflet 408 is fabricated using a suitable biocompatible material including, without limitation, a polyester, expanded polytetrafluoroethylene (ePTFE) or polyurethane material and combinations thereof. It is apparent to those skilled in the art and guided by the teachings herein provided that leaflets 408 may include any suitable biocompatible synthetic and/or biological material, which is suitable for implanting within the injured or diseased blood vessel.

One or more sensors are integrally coupled to at least one leaflet 408. In one embodiment, sensors 416 are covered by a thin layer of leaflet material. Sensors 416 are configured to detect or sense at least one structural characteristic of leaflets 408, such as a heart valve implant position, a leaflet wall stress, a leaflet wall strain, a leaflet wall tension and a leaflet temperature. Further, sensors 416 may be configured to detect a blood pressure through heart valve implant. In one embodiment, at least one sensor 416 is positioned with respect to a supra aortic aspect of leaflets 408 and at least one sensor 416 is positioned with respect to a subaortic aspect of leaflets 408 to facilitate detecting a pressure across the prosthetic implant. Additionally or alternatively, sensors 416 may be integrated at or near an edge of leaflets 408 and/or within a body portion of leaflets 408. Sensors 416 are integrated within leaflet 408 and configured to facilitate laminar flow at a corresponding inner surface of leaflet 408. Sensors 416 may include at least one piezoresistive sensor and/or at least one capacitive sensor. The sensors may be energized electromagnetically.

In one embodiment, sensors 416 are in signal communication with an external transmitter and receiver. Sensors 416 transmit signals representative of a structural characteristic of heart valve device 400. Data corresponding to the transmitted signals is gathered and complied to monitor leaflet wall tension, leaflet position and blood pressure, for example.

A power source is operatively coupled to sensors 416 and configured to power sensors 416. In a particular embodiment, the power source includes a radio frequency induction coil operatively coupled to sensors 416. In one embodiment, heart valve device 400 includes frame 402 positioned with respect to leaflets. Each leaflet 408 and at least one sensor 416 is coupled to frame 402. Sensors are coupled to frame 402 using a suitable coupling mechanism including, without limitation, soldering, gluing, sewing, welding, and heat bonding. In a particular embodiment, a plastic covering, enamel or epoxy is wrap around frame 402 to protect frame 402 and leaflets 408. An induction coil 422 is coupled to frame. In one embodiment, induction coil 422 is wrapped around at least a portion of frame 402 and/or is coupled to an inner aspect and/or an outer aspect of frame 402. Induction coil 422 is operatively coupled to each sensor 416 and configured to energize capacitor plates of sensor 416.

In one embodiment, sensors 416 are coupled to frame 402 to facilitate detecting or sensing a paravalvular leak. Further, coronary obstruction can be detected or sensed due to a proximity to the coronary ostia. The measurement of a trans-valvular gradient allows for a real-time monitoring of pressure change across the heart valve device as the valve is being deployed to provide an additional monitoring feature to facilitate evaluating valve deterioration during testing and after implantation. The monitoring of valve function during testing is limited by placement of the valve within a pressure-volume loop with strobe light visualization of valve leaflet coaptation. The placement of pressure sensors at the coaptation edges allows for the evaluation of the pressure at an edge of leaflet 408. The leaflet edge pressures created are similar to high and low pressure systems that develop at a trailing edge of an aircraft wing. Modifications to the leaflet edge geometry can be better monitored by the placement of ultraminature accelerometers, flow sensors and/or pressure sensors.

The trans-valvular gradients can be monitored in real time after implantation of the heart valve device to monitor wear on leaflets 408 and confirmed with echocardiography. The subaortic pressure sensors are capable of monitoring LVESP (left ventricular end systolic pressure) and LVEDP (left ventricular end diastolic pressure). The LVEDP is a marker for an injured and failing heart. A rise in the LVEDP above 25 mmHg is indicative of early heart failure. The increase in the trans-valvular gradient above 50 mmHg is indicative of developing aortic stenosis. The increase in the LVEDP with a concurrent decrease in the trans-valvular gradient is indicative of developing aortic regurgitation. If the LAP sensor is present and there is an increase in the LAP with a concurrent rise in the LVEDP then either a diagnosis of worsening heart failure can be made or a increasing mitral regurgitation along with worsening heart failure. If there is peripheral blood pressure sensor that indicates an increasing pulse pressure with an increasing LVEDP and lowering of the trans-valvular gradient then a consideration could be made for a diagnosis of severe aortic regurgitation.

In one embodiment, sensors are integrated into a cerebral spinal fluid (CSF) monitoring unit. In this embodiment, polymer-based sensors are integrated into a polymer-based shunt material such that a capacitor plate of sensor faces an inner lumen defined by the shunt. This capacitor plate is deflected by a change in pressure within the shunt as the CFS pressure changes. An algorithm controls monitoring of the shunt and includes a trigger that alarms to indicate that a shunt pressure should be checked, for example, if drainage of the CSF is obstructed. In alternative embodiment, CSF pressure is monitored by integrating at least one capacitive sensor into a wall of a ventricular shunt, such as an Omaya shunt. In a further alternative embodiment, polymer-based sensors are integrated into a tube configured for positioning within an inner ear to facilitate drainage of inner ear fluid that may build up under normal conditions and pathological conditions.

The above-described methods and apparatus provide a reliable method of monitoring an endoprosthesis after implantation into a body lumen. In one embodiment, the above-described methods and apparatus monitor the endoprosthesis by detecting and measuring pressures within a wall of the endoprosthesis. The pressure measurements are used to identify any changes to the structure of the endoprosthesis that may be indicative of an endoleak or damage to the endoprosthesis. By identify changes to the endoprosthesis, a more reliable indication of problems associated with the endoprosthesis is provided than would be when measuring characteristics of the body lumen wall. In addition, the above-described methods and apparatus can be used to detect and monitor various other attributes associated with the endoprosthesis and/or fluids flowing therethrough.

As used herein, an element or step recited in the singular and proceeded with the word “a” or “an” should be understood as not excluding plural said elements or steps, unless such exclusion is explicitly recited. Further, references to “one embodiment” of the present invention are not intended to be interpreted as excluding the existence of additional embodiments that also incorporate the recited features.

Although the apparatus and methods described herein are described in the context of monitoring an endoprosthesis with sensors, it is understood that the apparatus and methods are not limited to sensors or endoprosthetics. Likewise, the endoprosthetic and sensor components illustrated are not limited to the specific embodiments described herein, but rather, components of both the endoprosthesis and the sensors can be utilized independently and separately from other components described herein.

While the invention has been described in terms of various specific embodiments, those skilled in the art will recognize that the invention can be practiced with modification within the spirit and scope of the claims.

What is claimed is:

1. A method of monitoring an endoprosthesis for insertion into a body lumen, said method comprising,
implanting the endoprosthesis into the body lumen to exclude an aneurysmal sac in a vascular region; and
monitoring characteristics of the endoprosthesis using a plurality of sensors integrated with the endoprosthesis, wherein monitoring the characteristics comprises monitoring at least one of an endoprosthesis wall tension, an endoprosthesis circumference, an endoprosthesis diameter, a pressure on the luminal surface, and a pressure on the exterior surface.

2. A method in accordance with claim 1, wherein said monitoring the characteristics further includes monitoring an endoprosthesis temperature, endoprosthesis motion, and a pressure in a wall of the endoprosthesis.

3. A method in accordance with claim 1 further comprising detecting with the sensors at least one of a radial force associated with implantation and an accuracy of implantation.

4. A method in accordance with claim 1 further comprising detecting with the sensors a structural failure of the endoprosthesis.

5. A method in accordance with claim 1 further comprising measuring a constituent of the body lumen that is altered by the presence of blood flow through the endoprosthesis.

6. A method in accordance with claim 5 wherein the constituents comprise at least one of oxygen, enzymes, proteins, nutrients, and electrical potential.

7. A method in accordance with claim 1 further comprising:
providing a power source to the endoprosthesis;
providing at least one transmitter coupled to the endoprosthesis; and
transmitting signals with the at least one transmitter to a device external the body lumen.

8. A modular endoprosthesis for implantation in a body lumen to exclude an aneurysmal sac in a vascular region, said endoprosthesis comprising a plurality of sensors to monitor characteristics of said endoprosthesis, wherein said characteristics comprise at least one of an endoprosthesis wall tension, an endoprosthesis circumference, an endoprosthesis diameter, a pressure on the luminal surface, and a pressure on the exterior surface.

9. An endoprosthesis in accordance with claim 8 wherein said characteristics further comprise an endoprosthesis temperature, endoprosthesis motion, and a pressure in a wall of the endoprosthesis.

10. An endoprosthesis in accordance with claim 8 wherein said sensors are positioned to detect at least one of a radial force associated with implantation and an accuracy of implantation.

11. An endoprosthesis in accordance with claim 8 wherein said sensors are positioned to detect a structural failure of the endoprosthesis.

12. An endoprosthesis in accordance with claim 8 wherein said sensors are positioned to measure a constituent of the body lumen that is altered by the presence of blood flow through said endoprosthesis.

13. An endoprosthesis in accordance with claim 12 wherein the constituents comprise at least one of oxygen, enzymes, proteins, nutrients, and electrical potential.

14. An endoprosthesis in accordance with claim 8 further comprising a power source and at least one transmitter to transmit signals to a device external the body lumen.

15. A system for monitoring characteristics of an endoprosthesis, said system comprising:
a power source;
a modular endoprosthesis for implantation in a body lumen to exclude an aneurysmal sac in a vascular region, said endoprosthesis comprising:
a plurality of sensors to monitor characteristics of said endoprosthesis, wherein said characteristics comprise at least one of an endoprosthesis wall tension, an endoprosthesis circumference, an endoprosthesis diameter; and a pressure on the luminal surface, and a pressure on the exterior surface;
at least one transmitter to transmit signals indicative of said characteristics; and
a device external the body lumen to receive said transmitted signals.

16. A system in accordance with claim 15 wherein said characteristics further comprise an endoprosthesis temperature, endoprosthesis motion, and a pressure in a wall of the endoprosthesis.

17. A system in accordance with claim 15 wherein said sensors are positioned to detect at least one of a radial force associated with implantation and an accuracy of implantation.

18. A system in accordance with claim 15 wherein said sensors are positioned to detect a structural failure of the endoprosthesis.

19. A system in accordance with claim 15 wherein said sensors are positioned to measure a constituent of the body lumen that is altered by the presence of blood flow through said endoprosthesis.

20. A system in accordance with claim 19 wherein the constituents comprise at least one of oxygen, enzymes, proteins, nutrients, and electrical potential.

21. A prosthetic implant comprising:
a graft having a wall defining a passage; and
a plurality of sensors integrated with said graft, said plurality of sensors configured to detect at least one structural characteristic of said graft; and
a power source operatively coupled to said plurality of sensors and configured to provide power to said plurality of sensors.

22. A prosthetic implant in accordance with claim 21 wherein said power source further comprises a radio frequency induction coil operatively coupled to said plurality of sensors.

23. A prosthetic implant in accordance with claim 21 wherein said at least one structural characteristic further comprises at least one of a graft implant position, a graft temperature, a wall stress, a wall strain, a wall tension, an outer wall circumference, an inner wall circumference, an outer wall diameter, an inner wall diameter, a pressure on the luminal surface, and a pressure on the exterior surface.

24. A prosthetic implant in accordance with claim 21 wherein each sensor of said plurality of sensors further comprises one of a capacitive sensor and a piezoresistive sensor.

25. A prosthetic implant in accordance with claim 21 wherein at least one of said plurality of sensors is positioned within an inner surface of said wall.

26. A prosthetic implant in accordance with claim 21 wherein at least one sensor of said plurality of sensors is positioned within an outer surface of said wall.

27. A prosthetic implant in accordance with claim 21 wherein at least one sensor of said plurality of sensors is configured to detect at least one of a stress characteristic on said wall, a strain characteristic of said wall, a pressure on a luminal surface and a pressure on an exterior surface.

28. A prosthetic implant in accordance with claim 21 further comprising an induction coil, said induction coil comprising one of a planar coil, a spiral coil, a spiral coil having a ‘z’ configuration, and a vertical coil configuration.

29. A prosthetic implant in accordance with claim 21 further comprising:
a stent positioned with respect to said graft, said stent movable between a radially compressed configuration and a radially expanded configuration to support said graft within a body lumen; and
an induction coil wrapped around at least a portion of said stent.

30. A prosthetic implant in accordance with claim 21 wherein said plurality of sensors are configured to detect at least one of an intraluminal blood pressure, an intravascular blood pressure, a sac pressure and an aortic blood pressure.

31. A prosthetic implant in accordance with claim 21 wherein said plurality of sensors are configured about said graft in one of a helical pattern, a linear pattern, a star pattern, a circumferential pattern to facilitate monitoring an aneurysmal sac.

32. A prosthetic implant comprising:
a plurality of flexible leaflets cooperatively movable between an open position defining a passage and a closed position; and
at least one sensor integrated with at least one leaflet of said plurality of leaflets, said at least one sensor configured to detect at least one structural characteristic of said plurality of leaflets; and
a power source operatively coupled to said at least one sensor and configured to provide power to said at least one sensor.

33. A prosthetic implant in accordance with claim 32 wherein said power source further comprises a radio frequency coil operatively coupled to said at least one sensor.

34. A prosthetic implant in accordance with claim 32 further comprising:
a frame positioned with respect to said plurality of leaflets, each leaflet of said plurality of leaflets coupled to said frame, and at least one sensor coupled to said frame.

35. A prosthetic implant in accordance with claim 34 further comprising an induction coil coupled to said frame, said inductor coil operatively coupled to each said sensor of said at least one sensor and configured to energize capacitor plates of each said sensor.

36. A prosthetic implant in accordance with claim 32 wherein a first sensor of said at least one sensor is positioned with respect to a supra aortic aspect of said plurality of leaflets and a second sensor of said at least one sensor positioned with respect to a subaortic aspect of said plurality of leaflets to facilitate detecting a pressure across said prosthetic implant.
